(19) United States
(12) Patent Application Publication
(10) Pub. No.: US 2007/0190628 A1
(43) Pub. Date: Aug. 16, 2007

(54) Yeast food composition for fuel ethanol production

(76) Inventors: William Michael Ingledew, Saskatoon (CA); Eleonora Bellissimi, Saskatoon (CA)

Correspondence Address: KIRBY EADES GALE BAKER, BOX 3432, STATION D, OTTAWA, ON, K1P 6N9, CA

(21) Appl. No.: 11/350,761

(22) Filed: Feb. 10, 2006

(51) Int. Cl. C12P 7/06

(52) U.S. Cl. 435/161

(57) ABSTRACT

A method for producing a defined yeast food composition for supplementing a complex fermentation feedstock used for ethanol production. The method comprises selecting a complex fermentation feedstock and conducting a first series of steps wherein aliquots of the feedstock are supplemented with selected concentrations of selected individual nutritive elements. The supplemented aliquots are fermented and assessed for responses to the different nutritive elements whereby a preferred concentration for each element is chosen. A second series of steps is conducted to assess the effects of feedstock supplementation with selected combinations of selected individual nutritive elements on fermentation efficiency and production, wherefrom an optimal combination of elements is selected. The defined yeast food composition is the combination of elements selected with the method and preferably comprises a mixture of ionic salts plus urea. A method for ethanol production in a complex fermentation feedstock supplemented with the defined urea plus salts-based yeast food.

### FIELD OF THE INVENTION

This invention relates to fermentation production of ethanol. More particularly, this invention relates to the use of grain mashes for production of fuel-grade ethanol.

### BACKGROUND OF THE INVENTION

Gasoline combustion in vehicle engines is a major source of environmental pollution. Harmful emissions from motor vehicles caused by gasoline combustion can be significantly reduced by increasing the octane rating and the oxygen content of gasoline. Higher octane ratings and oxygen concentrations are accomplished by the process of reforming, also referred to as reformulating, the chemical composition of gasoline by the addition of “oxygenate” compounds. The most commonly and widely used oxygenate has been methyl tertiary butyl ether (MBTE) primarily because of its blending characteristics and for economic reasons. Reformulated gasoline has been in common use around the world since the late 1970s. However, it has become evident that MTBE by-products and residues are released into atmospheric environments during gasoline combustion or because of leakage from fuel storage tanks and/or surface spillage of petroleum products, and that these compounds accumulate in water and soil systems where they tend to be recalcitrant and persist for extended periods of time. It has been shown that exposure to MTBE and its by-products and residues significantly increases the occurrence of cancers and other serious illnesses and adverse health effects. Consequently, there are world-wide legislative pressures to reduce and/or eliminate the use of MTBE in gasoline reforming and to promote instead the development and use of renewable fuels and fuel additives such as ethanol.

Fuel ethanol is a preferred oxygenate substitute for MTBE because: (a) its octane rating is 113 compared to 109 for MTBE, (b) its oxygen content is 32% thereby easily providing the 2% final oxygen requirement for reformulated gasoline, and (c) it is clean-burning with no atmospheric polluting residues or by-products. It should be noted that MTBE is currently the preferred oxygenate for gasoline reforming due to its relative low cost. According to information on the US Govemment's Energy Information Administration's website (www.eia.doe.gov), reformulated gasoline contains about 11.5% MTBE (v/v US gal). The spot price for MTBE has fluctuated between $0.80-$1.60 USD per US gal since January 2000. Assuming a MTBE median price of $1.20 USD per US gal, the cost of MTBE per gallon of reformulated gasoline is approximately $0.14 USD. Because fuel ethanol has: (a) a higher octane rating than MTBE, and (b) a higher oxygen content, gasoline can be reformulated with 5.7% v/v ethanol. Accordingly, the cost of fuel ethanol should not exceed a median of $2.40 USD per gallon in order to provide an equivalent cost to MTBE for gasoline reformation.

Fermentation processes for production of ethanol are well-known and primarily use specifically selected and aerobically propagated strains of the yeast Saccharomyces cerevisiae as liquid slurried yeasts, compressed yeasts or active dry yeasts to produce certain grades and qualities of ethanol. The ethanol production process typically involves semi-anaerobic exploitation of yeast physiology via culturing a selected yeast strain through its entire life cycle which includes four distinct stages of growth i.e., (1) lag phase (also referred to as the conditioning phase), (2) exponential growth phase, (3) stationary phase, and if left long enough, (4) cell death phase. Such phases of growth, particularly the lag and exponential phases, may be repeated by culturing through several successive transfers in increasing volumes in a selected propagation medium during which time yeast cell numbers are multiplied to a target number of viable cells per mL after which, the culture is transferred into the selected production-scale fermentation medium wherein alcohol production occurs. The major ingredient in propagation and fermentation mash or media is also referred to as the feedstock. The media or mash may also contain precisely weighed mixtures of selected sugars, degraded starches, minerals, salts and enzymes; such media or mashes are referred to as complex or defined media depending on the sources of the media components and the complexity of the mixtures, and are used primarily for small-scale fermentations for research and development processes. Large-scale high-volume fermentation processes are typically conducted in complex fermentation media prepared from pulverized and processed plant materials including mashes produced from ground grains such as corn, wheat, barley rye and rice, chopped and/or pulverized vegetative plant materials such as sugar cane, sugar beets, or molasses, and high sugar and/or starch-containing waste streams from beverage production, food processing and other industrial processes.

It is known by those skilled in this art, that fermentation production of ethanol from complex media for human consumption can be enhanced by the addition of exogenous nutritive materials to the fermentation media prior to inoculation with yeast cells. Such exogenous nutritive materials are commonly referred to as “yeast foods” or “yeast nutrients” and typically comprise proprietary formulations of complex organic substrates such as yeast extracts, yeast autolysates, casein, and amino acids (Ingledew et al., 1986, Journal of the American Society of Brewing Chemists vol. 44, pages 166-171). Some yeast foods comprise complex organic substrates intermixed with selected defined salts and minerals. A common feature of known yeast foods is the incorporation of at least one or more of proteins, peptides or amino acids into their formulations even though only dipeptides and amino acids can be utilized by yeast cells.

Fermentation processes have been developed specifically for production of fuel-grade ethanol that is not necessarily suitable for human consumption. Such processes are based on producing maximal amounts of ethanol within the shortest possible fermentation times using the lowest cost feed stocks in order to reduce production costs as much as possible. Various strategies developed to accomplish these goals have focused on: (1) reducing the duration of the lag phase before the onset of the exponential growth phase, (2) providing and optimizing propagation and fermentation conditions during the lag and exponential phases to extend the duration of the period of time during which exponential but anaerobic cell growth occurs concomitantly with fermentation production of ethanol, and (3) conversion of grain mashes, industrial and food-processing waste streams into fuel alcohol fermentation feedstocks. A common strategy employed in the fuel ethanol production industry is to inoculate a large inoculum of active dry yeast cells into a relatively large fermentation vessel filled with a fermentation medium (typically a grain mash prepared from a ground grain, preferably corn). The inoculated vessel is maintained under semi-anaerobic or anaerobic conditions during which time the yeast cells are physiologically conditioned through a series of events known as “metabolic acclimitization” (Bellissimi et al., 2005, Proc. Biochem. Vol. 40, pp. 2205-2213). About 6-8 hrs after inoculation and metabolic acclimatization, the contents of the vessel are transferred into a larger vessel containing fresh fermentation medium, and culturing is continued under semi-anaerobic conditions and then anaerobic conditions in order to produce the highest possible cell yield e.g., ˜2.5×10⁸ cells/mL culture or ˜0.05 g cell dry wt/g substrate. Active dry yeast cells cultured under these conditions generally have reduced lag phases and typically commence exponential growth and ethanol production in shorter periods of time than if they had not been metabolically acclimatized. However, regardless of how quickly active dry yeast cells are conditioned in the lag phase and commence logarithmic growth, the period of yeast cell exponential growth in batch systems is short due to rapidly increasing CO₂ levels and rapidly decreasing concentrations of essential nutrients and the concomitant increases in concentrations of ethanol produced by the yeast cells which at about 10% to 13% v/v causes a deceleration of rapid yeast growth. The deceleration phase is also typically short and if nutrients are not replenished in the fermentation medium during this time period, the yeast cells will move into an extended period of stationary phase during which the yeast cells do not reproduce but remain metabolically active and continue producing ethanol although at lower rates than when the yeast cells are actively growing. In such systems, grain mashes containing dissolved solid concentrations of about 24% prior to inoculation with active yeast cells typically produce about 10%-12% fuel grade ethanol. It is known that grain mashes are deficient in nitrogen sources that are utilizable by yeasts. Consequently, grain mashes may be supplemented with nitrogen in the form of urea prior to inoculation to extend the duration of the exponential growth phase during which time ethanol is more rapidly produced. However, while urea supplementation increases yeast growth rates and their protein contents, it also reduces the fermentation time required for the yeast cells to consume the dissolved solids from the fermentation medium—yet greater amounts ethanol are not produced.

### SUMMARY OF THE INVENTION

The exemplary embodiments of the present invention, at least in preferred forms, are directed to the fermentation production of ethanol by yeast cells.

According to one specific embodiment of the present invention, there is provided a method for producing a defined yeast food composition for supplementing a complex fermentation feedstock, the feedstock being useful for fermentation production of ethanol therein. The method comprises selecting a complex fermentation feedstock; conducting a first series of steps wherein aliquots of the feedstock are supplemented with selected concentrations of selected individual nutritive elements, the supplemented aliquots being inoculated with a yeast culture and fermentations being conducted therewith, assessing the fermentations for effects therein of the concentrations of selected individual nutritive elements, and selecting an optimal concentration of each individual nutritive element therefrom. The method then continues by conducting a second series of steps wherein aliquots of the feedstock are supplemented with selected combinations of said individual nutritive elements, the supplemented aliquots being inoculated with a yeast culture and fermentations being conducted therewith, assessing the fermentations for effects therein of the combinations, selecting an optimal combination of individual nutritive elements therefrom; and preparing a yeast food composition containing the optimal concentrations.

According to one aspect of the invention, the culture medium selected for the first series of steps is a complex fermentation feedstock selected from the group consisting of grain mashes, pulverized and/or processed and/or digested vegetative plant materials, enzymatically and/or chemically digested cellulosic and/or lignocellulosic wastestreams from plant and/or wood processing operations, and high-carbohydrate containing wastestreams from beverage or food or industrial production processes. In a preferred form, the complex fermentation feedstock is a grain mash. In a further preferred form, the grain mash is a corn mash.

According to another aspect of the invention, the fermentation feedstock selected for the second series of steps is a complex fermentation feedstock selected from the group consisting of grain mashes, pulverized and/or processed vegetative plant materials, ezymatically and/or chemically digested cellulosic and/or lignocellulosic wastestreams from plant and/or wood processing operations, and high-carbohydrate containing wastestreams from beverage or food or industrial production processes. In a preferred form, the industrial fermentation feedstock is a grain mash. In a further preferred form, the grain mash is a corn mash.

According to yet another aspect of the invention, the selected individual essential nutrient elements assessed in the first series of steps are selected from the group consisting of carbon, oxygen, nitrogen, phosphorus, potassium, magnesium, sulphur, zinc, iron, manganese, trace mineral metals, and vitamins. In a preferred form, the individual essential nutrient elements are selected from a group of minerals consisting of comprise the group consisting of nitrogen, phosphorus, potassium, magnesium, sulphur, zinc, iron, manganese, and trace mineral metals. In a further preferred form, the selected essential nutrient elements comprise the group consisting of nitrogen, phosphorus, magnesium, sulphur and zinc.

According to another specific embodiment of the present invention, there is provided a defined yeast food composition for supplementing fermentation feedstocks for ethanol production. The defined yeast food composition comprises a mixture of selected individual salts blended together, said individual salts selected to provide essential nutritive elements to yeast cells during fermentive production of ethanol. The defined salts-based yeast food composition is preferably added to fermentation feedstocks prior to inoculation with yeast cells. Optionally if so desired, the defined salts-based yeast food composition of the present invention may be added to fermentation feedstocks during fermentation production of ethanol.

According to one aspect of the invention, the individual salts are selected from the group consisting of nitrogen, phosphorus, potassium, magnesium, sulphur, zinc, iron, manganese, and trace mineral metals. In a preferred form, the selected individual salts comprise the group consisting of nitrogen salts, phosphorus salts, magnesium salts, sulphur salts and zinc salts.

According to yet another specific embodiment of the present invention, there is provided a method for producing ethanol wherein a fermentation feedstock is supplemented with a defined yeast food composition comprising a mixture of salts, said supplemented fermentation feedstock then inoculated with a selected yeast cell mass after which the yeast cell mass is cultured under semi-anaerobic or anaerobic conditions.

According to one aspect, the fermentation feedstock is a complex fermentation feedstock selected from the group consisting of grain mashes, pulverized and/or processed vegetative plant materials, ezymatically and/or chemically digested cellulosic and/or lignocellulosic wastestreams from plant and/or wood processing operations, and high-carbohydrate containing wastestreams from beverage or food or industrial production processes. In a preferred form, the industrial fermentation feedstock is a grain mash. In a further preferred form, the grain mash is a corn mash.

According to another aspect, the defined yeast food composition comprises a mixture of salts selected by a process comprising (1) a first series of steps wherein the optimal concentrations of selected individual essential nutrient elements contained within actively metabolising yeast cells cultured in a selected culture medium under semi-anaerobic or anaerobic conditions, are determined, said yeast cells sampled during exponential growth, (2) a second series of steps for assessing the effects of multiple combinations of selected individual salts comprising said individual essential nutrient elements, on the rates of metabolism and ethanol production by yeast cells sampled during exponential growth in a selected fermentation feedstock, and for determining optimal concentration ranges of each selected nutrient element in said salt combinations, and (3) a third series of steps wherein selected optimal concentrations of the selected individual salts are blended together to optimally formulate yeast food compositions suitable for supplementing fermentation feedstocks for production of ethanol. Alternatively, the selected individual salts may be separately added directly into a fermentation feedstock before the feedstock is inoculated with a yeast culture. In a preferred form, the fermentation feedstock is a complex fermentation feedstock selected from the group consisting of grain mashes, pulverized and/or processed vegetative plant materials, enzymatically and/or chemically digested cellulosic and/or lignocellulosic wastestreams from plant and/or wood processing operations, and high-carbohydrate containing wastestreams from beverage or food or industrial production processes. In another preferred form, the yeast food composition is useful for supplementing a complex fermentation feedstock for production of fuel grade ethanol.

According to yet another aspect, the yeast cell mass is selected from the group comprising an active dry yeast cell mass, a compressed yeast, and a yeast slurry. It is preferred that the yeast cell mass is a commercially available active dry yeast.

### BRIEF DESCRIPTION OF THE DRAWINGS

The present invention will be described in conjunction with reference to the following drawings, in which:

FIG. 1 is a graph showing the effects of temperature on fermentation of corn mash by a commercial active dry yeast “z”;

FIG. 2 is a graph showing the effects of urea supplementation of corn mash on biomass production and fermentation rates by active dry yeast “z”. Open squares show the effects on fermentation rates (g of utilized dissolved solids/100 mL/h). Closed squares show the effects on maximum viable cell numbers (CFU/mL). Standard error bars are included in the results. If error bars are not seen, they are contained within the data points;

FIG. 3 is a graph showing the effects of phosphorus supplementation of corn mash on biomass production and fermentation rates by active dry yeast “z”. Open squares show the effects on fermentation rates (g of utilized dissolved solids/100 mL/h). Closed squares show the effects on maximum viable cell numbers (CFU/mL). If error bars are not visible, they are contained within the data points;

FIG. 4 is a graph showing the effects of magnesium supplementation of corn mash on biomass production and fermentation rates by active dry yeast “z”. Open squares show the effects on fermentation rates (g of utilized dissolved solids/100 mL/h). Closed squares show the effects on maximum viable cell numbers (CFU/mL). If error bars are not visible, they are contained within the data points;

FIG. 5 is a graph showing the effects of sulfur supplementation of corn mash on biomass production and fermentation rates by active dry yeast “z”. Open squares show the effects on fermentation rates (g of utilized dissolved solids/100 mL/h). Closed squares show the effects on maximum viable cell numbers (CFU/mL). If error bars are not visible, they are contained within the data points;

FIG. 6 is a graph showing the effects of zinc supplementation of corn mash on biomass production and fermentation rates by active dry yeast “z”. Open squares show the effects on fermentation rates (g of utilized dissolved solids/100 mL/h). Closed squares show the effects on maximum viable cell numbers (CFU/mL). If error bars are not visible, they are contained within the data points;

FIG. 7 is a half-normal plot of the interactive effects of combined individual nutritive salts in corn mash on the maximum numbers of viable cells (cell number X 10⁸) produced by active dry yeast “z”;

FIG. 8 is a half-normal plot of the interactive effects of combined individual nutritive salts in corn mash on the maximum numbers of viable cells (cell number X 10⁸) produced by active dry yeast “z”;

FIG. 9 is a graph showing the effects of a defined yeast food composition of the present invention added to a normal gravity corn mash on fermentation rates by active dry yeast “z”. Open squares show fermentation in corn mash supplemented with 8 mM urea. Closed circles show fermentation rates in corn mash supplemented with 1% (w/v) yeast extract. Closed triangles show fermentation rates in corn mash supplemented with 8 mM urea plus the defined yeast food of the present invention;

FIG. 10 is a graph showing the effects of a defined yeast food composition of the present invention added to a very high gravity corn mash on fermentation rates by active dry yeast “z”. Open squares show fermentation in corn mash supplemented with 16 mM urea. Closed circles show fermentation rates in corn mash supplemented with 1% (w/v) yeast extract. Closed triangles show fermentation rates in corn mash supplemented with 16 mM urea plus the defined yeast food of the present invention; and

FIG. 11 is a graph showing the effects of a defined yeast food composition of the present invention on fermentation rates and ethanol production by active dry yeast “z” in a scaled-up batch fermentation process using normal gravity corn mash. Open squares show fermentation rates i.e., disappearance of dissolved solid (g/100 mL) in corn mash supplemented with 8 mM urea while closed squares show fermentation rates in corn mash supplemented with the defined yeast food of the present invention. Open circles show concomitant ethanol production (% v/v) in corn mash supplemented with 8 mM urea while closed circles show ethanol production (% v/v) in corn mash supplemented with the defined yeast food of the present invention.

### DETAILED DESCRIPTION OF THE INVENTION

Exemplary embodiments of the present invention as disclosed herein provide defined urea plus salts-based yeast food compositions for supplementing complex fermentation feedstocks used for large-scale fermentation production of ethanol, methods for producing said defined urea plus salts-based yeast food compositions, and methods for use of said defined urea plus salts-based yeast food compositions.

Complex fermentation feedstocks contain significant quantities of polymeric carbohydrates derived from plant materials and/or plant processing wastestreams and/or industrial wastestreams. Illustrative examples of such complex organic substrates include grain mashes, pulverized and/or processed vegetative plant materials, ezymatically and/or chemically digested cellulosic and/or lignocellulosic wastestreams from plant and/or wood processing operations, and high-carbohydrate containing wastestreams from beverage or food or industrial production processes. Such complex organic substrates are particularly useful for production of fuel-grade ethanol. In particular, grain mashes produced from ground corn grain are commonly used for fuel-grade ethanol production in North America. The composition of corn grain (dry) on average comprises about 71.7% starch, 9.5% proteins, 8.8% pentosan and monosaccharide sugars, 4.3% fats, 3.3% cellulose and lignins, and 1.4% ash (Watson, 1999, in Corn: Chemistry and Technology, Watson and Ramstad Eds., Chapter 3, pp. 53-83, American Association of Cereal Chemists; Wright, 1999, in Corn: Chemistry and Technology, Watson and Ramstad Eds., Chapter 15, pp. 447-478, American Association of Cereal Chemists). The 1.4% ash component of grain comprises mineral nutrients which include phosphorus (˜0.29% of the grain dry wt.), potassium (˜0.37%), magnesium (˜0.14%), and sulphur (˜0.12%), as well as trace amounts of essential micronutrients such as iron, zinc, manganese. It is clear that while grain mash is rich in starch and sugar-containing molecules, it is deficient in yeast-utilizable nitrogen, phosphorus, sulfur, and nutritive mineral elements.

Actively metabolizing yeast cells require balanced carbohydrate, oxygen, nitrogen, phosphorus, sulfur and mineral nutrition for optimal metabolic growth, cell reproduction, and fermentation production of ethanol. Phosphorus is involved in many metabolic reactions including sugar metabolism, lipid synthesis, cellular membrane function and production of nucleic acids. It is a key component of production and storage of cellular energy as ATP. Uptake of phosphorus by metabolizing yeast cells is highly dependent on its form (e.g., in phytate molecules vs. phosphate ions, the presence of other elements including magnesium and potassium, as well as the intra and extracellular pH levels and the presence of a fermentable substrate. Sulfur is required primarily for the biosynthesis of the sulfur-containing amino acids, methionine and cysteine/cystine as well as the tripeptide glutathione, and vitamins for coenzymes (acetyl CoA) used in metabolism. Magnesium ions are essential for fermentative enzymes used in anaerobic metabolism of sugars by yeast cells. Magnesium ions are also involved in stress alleviation, and in regulating cellular levels of other ions. Zinc is an essential cofactor of enzymes directly involved in fermentation production of ethanol. Zinc ions also stimulate active uptake of maltose and maltotriose by yeast cells.

The speed with which complex fermentation feedstocks are converted into ethanol and the amounts of ethanol produced are likely limited by one or more of physical culture conditions (i.e., temperature, aeration etc.), the availability of suitable carbohydrate substrates and essential nutritive elements, and the metabolic efficiency of the selected yeast culture. A wide variety of commercial liquid yeast slurries, compressed yeasts, and active dry yeasts are known and readily available. Optimized physical culture conditions for large-scale fermentation production of ethanol are also known, e.g., as reviewed by Kelsall et al (1999) in The Alcohol Textbook, 3^{rd} Ed., pages 7-38, Alltech Inc. Furthermore, it has been shown that supplementation of grain mashes with nitrogen in the form of urea can significantly increase fermentation rates and reduce fermentation times in complex fermentation feed streams (i.e., Jones et al., 1994, Proc. Biochem. Vol. 19, pages 483499; Wang et al., 1998, Appl. Biochem. Biotechnol. Vol. 69, pages 157-175). However, it is not known if supplementation with one or more mineral elements in combination with urea will benefit large-scale fermentations of complex fermentation feedstocks.

The inventors have surprisingly discovered a method for producing defined urea plus salts-based yeast food compositions for concurrently significantly increasing fermentation rates and reducing overall fermentation time, said yeast food compositions comprising combinations of salts of selected nutritive mineral elements with urea. The method requires the availability of benchmark information regarding the concentrations of individual nutritive elements contained within metabolically vigorous yeast cells growing at maximal exponential growth rates under anaerobic conditions wherein nutrients are not likely limiting. Illustrative examples of nutritive elements which may serve as benchmarks include nitrogen, potassium, phosphorus, magnesium, sulphur and various trace mineral elements. Such benchmark information may be obtained from publicly available sources e.g., as reviewed by Ingledew (1999) in Proceedings of Alltech's 15^{th} Annual Symposium, pp 27-47, Nottingham University Press. Alternatively, such benchmark information may be generated by culturing a selected yeast under optimal nutritional and cultural conditions, harvesting metabolically vigorous yeast cells growing at maximal exponential growth rates therein, and analyzing said yeast cells to determine the concentrations of selected individual nutritive elements therein.

The first step of the method is the determination of the amount of nitrogen supplementation required in a selected complex fermentation feedstock in order to maximize fermentation rates therein while minimizing fermentation time. The first step is based on the assumption that the nitrogen concentration of metabolically vigorous yeast cells growing at maximal exponential growth rates under anaerobic conditions wherein nutrients are not limiting is ˜6%. A target cell count for metabolically vigorous yeast cells growing at maximal exponential growth rates under anaerobic conditions was set at 3×10⁸ cells/mL culture medium. Assuming that ˜4.87×10¹⁰ total yeast cells would be equivalent to 1 gram dry weight per Ingledew, 1999, in The Alcohol Textbook, 3^{rd} Ed., pages 49-87, Alltech Inc., 3.0×10⁸ cells/mL would weigh approximately 6.16 g/L. To grow 6.16 g of cells/L, ˜0.37 g of supplemental nitrogen per L of medium would be required. Assuming that urea (MW: 60.02) is a preferred nitrogen source, therefore (0.37 g×60.02)/(2×14) or 0.8 g/L of urea would be added to the selected complex fermentation feedstock (assuming no other usable nitrogen in the medium). To determine the optimum level of urea i.e., nitrogen needed, a range of concentrations should then be examined around this value. After the optimum nitrogen concentration has been determined in the first step, that nitrogen concentration is set as the baseline for the remaining two steps of the method of the present invention.

The second step of the method comprises a series of steps wherein concentrations of salts of selected individual nutritive elements, are varied around the benchmark concentration for each nutritive element, and are individually added to the selected complex fermentation feedstock that has been supplemented with the optimum nitrogen concentration determined in step 1 of the present method, thereby enabling identification of the optimum concentration of each selected nutritive element for increasing fermentation rates and/or decreasing fermentation times and/or increasing ethanol production within the selected complex fermentation feedstock.

The third step of the method comprises a series of steps wherein combinations of salts of selected individual nutritive elements wherein the concentration of each selected individual nutritive element is varied, are added to the selected complex fermentation feedstock that has been supplemented with the optimum nitrogen concentration determined in step 1 of the present method, thereby enabling identification and selection of an optimum combination of selected nutritive elements for increasing fermentation rates and/or decreasing fermentation times and/or increasing ethanol production within the selected complex fermentation feedstock. Such selected optimum combinations of salts of the selected nutritive elements comprise the defined urea plus salts-based yeast food compositions of the present invention.

The defined urea plus salts-based yeast food compositions of the present invention can be provided as mixtures containing therein the concentrations of each selected individual nutritive element from the optimum combinations selected in the third step of the method of the present invention. Alternatively, the selected concentration of each selected individual nutritive element from the optimum combinations selected in the third step, may be added and mixed directly into the selected complex fermentation feedstock prior to inoculation of the feedstock with a yeast culture.

It is preferable that the defined urea plus salts-based yeast food compositions of the present invention are added to selected complex fermentation feedstocks prior to inoculation with a yeast culture for large-scale fermentation production of ethanol. However, the yeast food composition may also be added to selected complex fermentation feedstocks during any fermentation process. The optimal time for adding the yeast food composition is when the yeast cells begin to grow in the fermentation feedstock.

The defined urea plus salts-based yeast food compositions, methods for producing said yeast food compositions, and methods for the use of said yeast food compositions of the present invention are described in more detail in the following examples which are intended to be exemplary of the invention and are not intended to be limiting.

### EXAMPLE 1

Determination of Optimum Levels of Nutritive Elements for Maximal Yeast Cell Metabolic Activity and Fermentation During Exponential Growth.

(a) Fermentation Feedstock (Normal Gravity Corn Mash):

Normal gravity corn mash was prepared using ground #2 yellow dent corn obtained from Commercial Alcohols Inc. (275 Bloomfield Road, Chatham, Ontario, Canada N7M 5J5). Eighty-five percent (w/w) of this ground corn had a particle size less than 20 mesh. One part by weight of ground corn was dispersed into three parts of 50° C., sterile dH₂O containing 1 mM CaCl₂.2H₂O. To minimize the development of excessive viscosity during gelatinization, 1.25 mL of high temperature (HT-2X) α-amylase such as was supplied by Alltech Inc. in Nicholasville, Ky., USA (HT-2X α-amylase is no longer available from Alltech), was added per litre of mash as the ground corn was dispersed. It should be noted that if Ca-independent alpha amylases are used such as Termamyl® SC supplied by Novozymes North America in Franklinton, N.C., USA, then the 1 mM CaCl₂.2H₂O is not required. Following the addition of the ground corn and a five-minute mixing period to produce a homogenous mixture, the temperature was increased until the mash reached 94° C. where it was held at this temperature for 60 min with continued stirring. The temperature of the gelatinized starch was then reduced to 80° C. and liquefied by a second dose of HT-2X αx-amylase (1.25 mL/L) with continued stirring for 30 min. Sterile Celstir® (Wheaton Scientific, Millville, N.J.) fermentor vessels were filled with 1 L aliquots of this mash. To each fermentor vessel, filter-sterilized (25 mm, 0.2 μm Acrodisc® syringe filter, Pall Corporation, Ann Arbor, Mich.) urea (Fisher Scientific, Co., NJ), was added to achieve a final concentration of 8 mM. Cold-sterilization of the mash was assured by the addition of 100 μL of diethyl pyrocarbonate (DEPC, Sigma Chemical Co.) per litre of mash mixing the mash mechanically (IKA-Labortechnik, Staufen, Germany) for 5-10 min followed by cooling the fermentor vessels at 4° C. cold room for ˜72 hrs. The fermentor vessels were then removed from the cold room, connected to 30° C. water bath circulators and mixed. Glucoamylase in the form of Allcholase II 400 supplied by Alltech Inc. was then added (1.5 mL/L mash) 30 min prior to yeast inoculation to initiate saccharification of the dextrins formed during gelatinization and liquefaction (it should be noted that Allcholase II 400 is no longer available and any glucoamylase may be used). The mash thus prepared contained 19-20 g of dissolved solids/100 mL of mash as determined by a digital density meter analyzer (model DMA-45, Anton Paar KG, Graz, Austria) maintained at 20° C.

(b) Yeast Culture and Propagation:

Eleven grams of a commercially available active dry yeast, designated as “z”, were rehydrated by dispersal in 99 mL of preheated 38° C., 0.1% (w/v) sterile water and incubation for 20 min with periodic shaking in a 38° C. water bath. Fermentor vessels containing normal density corn mash were prepared as described above and inoculated with sufficient active yeast to provide an initial viable cell count of 1×10⁷ cells per mL mash. The optimal fermentation temperature for the active dry yeast “z” was determined by conducting fermentations at 30°, 34°, 36°, 38°, and 40° C.

(c) Determination of the Effects of Individual Nutritive Elements on Yeast Cell Biomass Production and Fermentation Rates:

The effects of individual elements on yeast cell biomass production and fermentation in a fermentation feedstock were assessed by separately supplementing of corn mash with nitrogen, phosphorus, magnesium, sulphur and zinc. Nitrogen effects were assessed in corn mashes by the additions of urea to provide final concentrations in the mash of 0, 1, 2, 4, 8, 16 and 32 mM urea/mL. Phosphorus effects were assessed in corn mashes amended with 16 mM urea, by the additions of (NH₄)₂HPO₄ (BDH Chemicals Inc, Toronto, Ont., MW: 132.06) to provide final concentrations of 0, 0.1, 0.2, 0.4, 0.6, and 0.8 g of (NH₄)₂HPO₄/L of mash. Magnesium effects were assessed in corn mashes amended with 16 mM urea, by the additions of MgSO₄.7H₂O (BDH Chemicals Inc., MW. 246.48) to provide final concentrations of 0, 0.025, 0.049, 0.074, 0.099, 0.148, 0.197 g of MgSO₄.7H₂O/L of mash. Sulfur effects were assessed in corn mashes amended with 16 mM urea, by the additions of (NH₄)₂SO₄ (BDH Chemicals Inc., FW: 132.13) to provide final concentrations of 0, 0.01, 0.02, 0.04, 0.08, and 0.16 g of (NH₄)₂SO₄/L of mash. Zinc effects were assessed in corn mashes amended with 16 mM urea, by the additions of ZnSO₄.7H₂O (BDH Chemicals Inc., MW. 287.54) to provide final concentrations of 0, 0.287, 0.575, 1.15, 1.73, 2.30 mg of ZnSO₄.7H₂O/L of mash. Each of the above supplements was done individually and examined for its effects on yeast growth and fermentation. Each corn mash preparation amended with a nutrient element as described above was inoculated with sufficient rehydrated active dry yeast to provide 1×10⁷ viable yeast cells/mL of mash at the start of each study.

The rates of yeast biomass production were determined by following the method described by Ingledew et al. in the Journal of the American Society of Brewing Chemists vol. 38, pages 125-129 (1980) whereby each nutrient-amended corn mash was sampled at set time intervals throughout the course of each study. A dilution series prepared from each sample was then filtered in triplicate through sterile membrane filters. The membrane filters were then placed on YPD agar plates and incubated for 3 days at 28° C. after which, the numbers of colonies formed were counted to determine numbers of viable yeast cells/mL of fermentation broth. Generation times were calculated using the following formulae:

$${μ}_{max} = \frac{log N - log {N}_{o}}{t - {t}_{o}} \times 2.303 \\ G = \frac{ln 2}{{μ}_{max}} \qquad ( 1 )$$

where G is the generation time, μ_{MAX} is the maximum specific growth rate, Nₒ and tₒ are yeast cell numbers and time in early log phase, whereas N and t are cell numbers and time in mid to late log phase.

The fermentation rate in each nutrient-amended corn mash was determined by following the disappearance of dissolved solids by analysis of each sample collected for determination of yeast biomass production, with a digital density meter analyzer (model DMA-45, Anton Paar KG, Graz, Austria) maintained at 20° C.

The amounts of ethanol produced during fermentation of each nutrient-amended corn mash were determined by measuring the amount of NADH+H⁺ produced after ethanol was converted to acetaldehyde by alcohol dehydrogenase (ADH; Sigma Chemical Co.). An aliquot of each sample was collected for determination of yeast biomass production and diluted to 1:500. To screw cap test tubes 0.1 mL of the diluted 0 to 24 h samples, 3 mL of NAD buffer, (1.194 mg NAD in 3 mL of glycine buffer) and 0.05 mL of ADH enzyme, (56 mg ADH per 5 mL of dH₂O) were added resulting in a total cuvette volume of 3.15 mL. For the 32 h, 48 h and 72 h samples, 0.05 mL of the sample was combined with 0.05 mL of dH₂O, 3 mL of NAD⁺, and 0.05 mL of ADH enzyme. The absorbance of each tube was then measured at 340 nm after 15 min of incubation using water as the blank. To determine the amount of ethanol produced, a standard curve (ethanol concentration versus absorbance) was prepared using a standard 10% (w/v) ethanol preparation diluted to 1:500 and then diluted further to produce range of ethanol concentrations from 0 to 20 μg in the assay above. The linear equation (y=mx+b) obtained from the plot was used to calculate the amount of ethanol in the test solutions, where y was the absorbance and x was the amount (μg) of ethanol. Based on the μg of ethanol determined from the standard curve, this value was converted to μg/mL and then to % v/v of ethanol using the formula:

$$\% v / v = \frac{x ( μg ) \cdot 100 mL \cdot 500 ( dilution factor ) \cdot 1 g}{1 \times {10}^{6} μg} \cdot ( 1 / 0.789 ) \qquad ( 2 )$$

where (1/0.789) is the conversion unit used to convert weight/volume of ethanol to volume/volume.

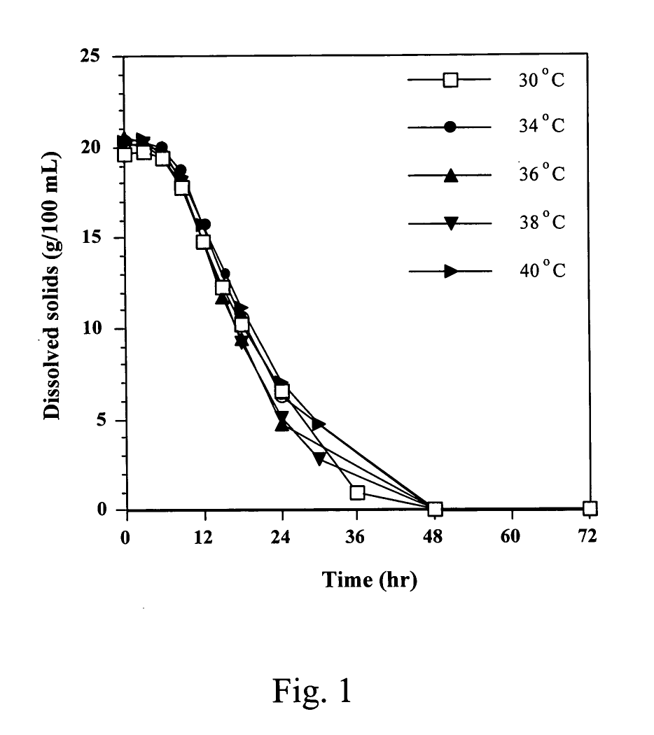

As shown in FIG. 1, the rates of fermentation by the active dry yeast “z” of normal gravity corn mash which did not receive any nutrient supplementations, were not significantly affected by temperatures in the range of 30° C.-38° C. Approximately 48 hrs were required for fermentation to be completed as measured by the disappearance of dissolved solids. Subsequent studies were conducted with fermentations maintained at 30° C.

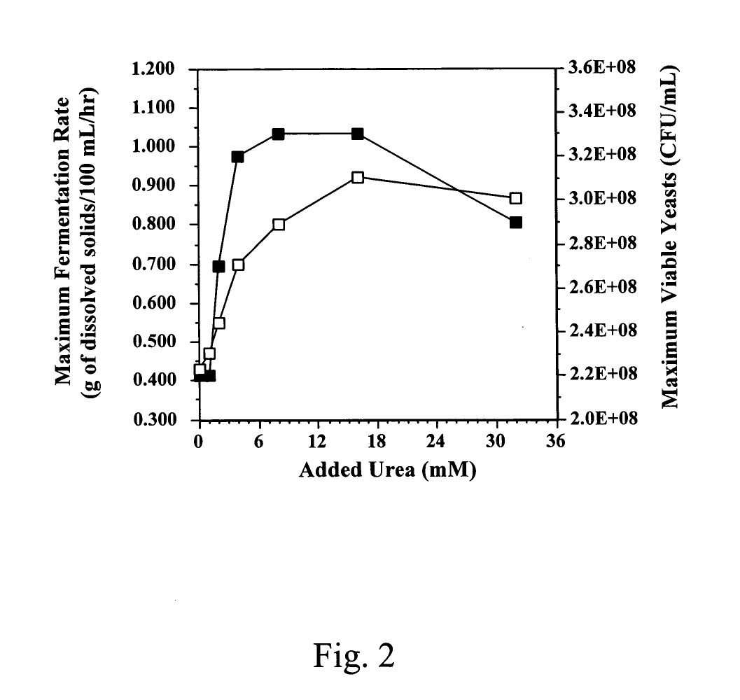

Supplementation of corn mash with urea concentrations in the range of 16-32 mM increased the rate of dissolved solids utilization approximately 2-fold (FIG. 2; refer to data with open squares). Although urea concentrations were tested up to 32 mM, the maximum fermentation rate was observed with the 16 mM supplementation rate. The highest yeast cell counts were achieved with corn mash supplemented with 8 mM and 16 mM of urea (FIG. 2; refer to data with closed squares); cell numbers were seen to increase by 50%, from 2.2×10⁸ CFU/mL to 3.3×10⁸ CFU/mL. Therefore, subsequent studies were conducted with corn mash supplemented with 16 mM of urea.

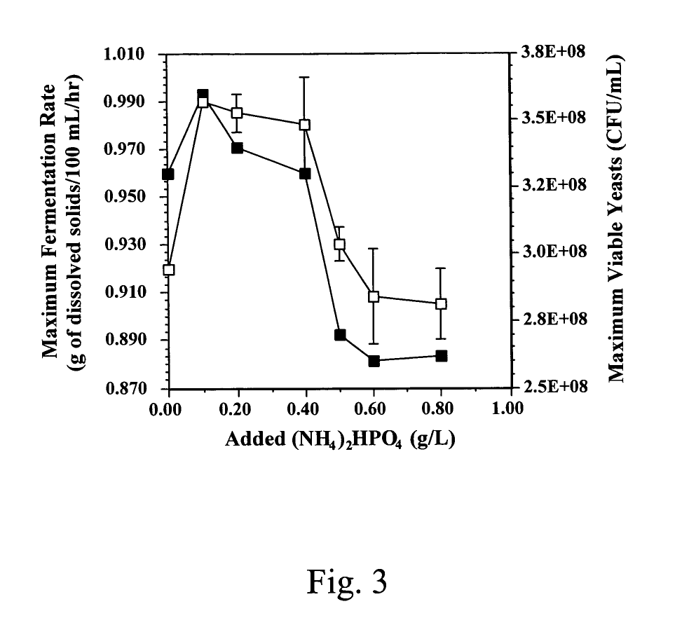

The effects of phosphate supplementation on fermentation production of ethanol were assessed in corn mash supplemented with 16 mM urea. Based on the data shown in FIG. 2, it was assumed that the ammonium-ion component of the phosphorus source used in this study i.e., (NH₄)₂HPO₄, would not influence nitrogen benefits on fermentation rate as provided by the optimized 16 mM urea supplement. Increasing phosphate supplementation of corn mash with 0.1 g/L to 0.4 g/L (NH₄)₂HPO₄ significantly increased the rates of dissolved solids utilization (FIG. 3; refer to data with open squares). However, increasing (NH₄)₂HPO₄ concentrations above 0.4 g/L resulted in decreased fermentation rates (FIG. 3; refer to data with open squares) and decreased cell yields (FIG. 3; refer to data with closed squares).

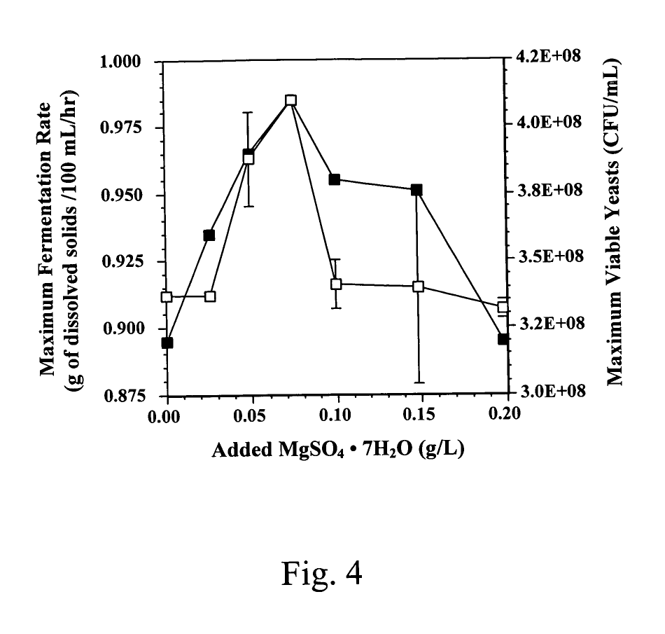

The effects of magnesium supplementation on fermentation production of ethanol were assessed in corn mash supplemented with 16 mM urea. Based on the Mg²⁺ content of anaerobically grown cells, it was determined that the optimum level required to support the growth of 6.16 g of cells/L was ˜0.144 g/L of MgSO₄.7H₂O (0.6 mM Mg²⁺). MgSO₄.7H₂O was added to the prepared corn mash to obtain final concentrations of 0.0248 g/L to 0.197 g/L. It was observed that concentrations exceeding 0.074 g/L MgSO₄.7H₂O (0.3 mM Mg²⁺) did not further benefit fermentation but actually decreased fermentation rate (FIG. 4; refer to data with open squares). Similar results were seen for the total maximum cell yields that could be achieved (FIG. 4; refer to data with closed squares).

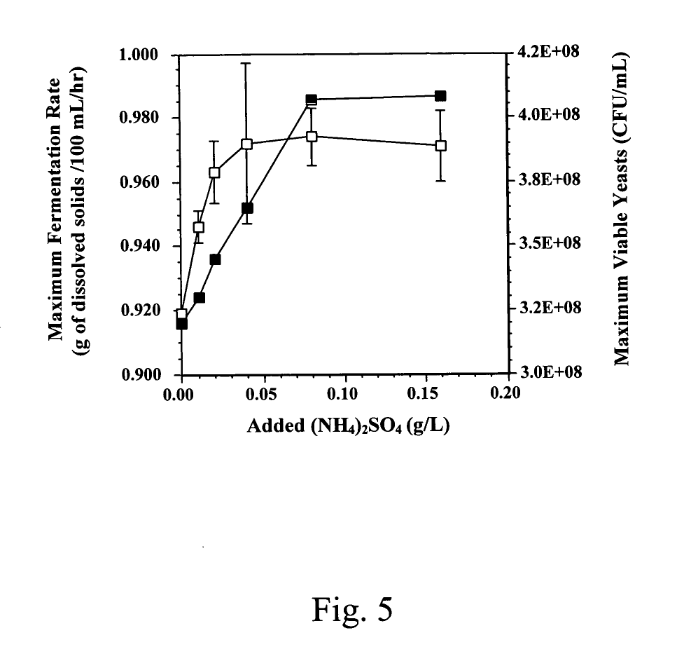

The effects of sulfur supplementation on fermentation production of ethanol were assessed in corn mash supplemented with 16 mM urea. (NH₄)₂SO₄ concentrations of 0.01 g/L to 0.16 g/L were tested. The data in FIG. 5 (refer to data with open squares) show that while increasing (NH₄)₂SO₄ concentrations above 0.04 g/L did not further benefit the rate of fermentation, yeast cell yields increased 3.3×10⁸/mL to 4×10⁸/mL (refer to data with closed squares). These data show that sulfur concentrations from 0.03 g/L (0.04 g/L of (NH₄)₂SO₄) to 0.1 g/L (0.16 g/L of (NH₄)₂SO₄) were optimal for overall cell growth in the corn mash medium supplemented with 16 mM urea.

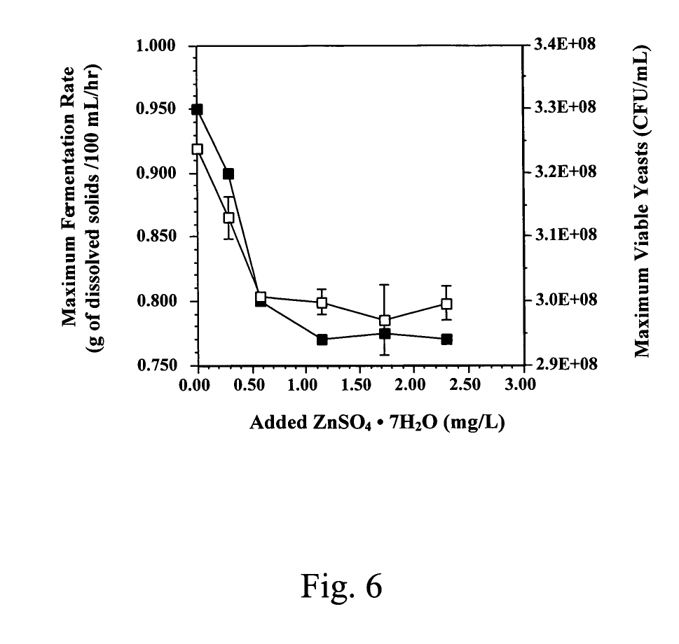

The effects of zinc supplementation on fermentation production of ethanol were assessed in corn mash supplemented with 16 mM urea. Zn²⁺ is an essential ion required for many key reactions of fermentative metabolism and yeast growth. However, the data in FIG. 6 show that supplementation of corn mash with ZnSO₄.7H₂O at concentrations ranging from 0.287 mg/L to 2.30 mg/L (1 to 8 μM) had negative effects on fermentation rates (refer to data with open squares) and numbers of yeast cells produced (refer to data with open squares).

### EXAMPLE 2

Assessment of Combinations of N, P, S, Mg²⁺ and Zn²⁺ on Fermentations of Corn Mash by a Commercial Active Dry Yeast.

A 2^{K} single replicate factorial experiment was designed following the approach described by Kennedy et al. Journal of Industrial Microbiology and Biotechnology vol. 23 pages 456-475 (1999) in reference to the principles outlined by Montgomery (1989, Design and Analysis of Experiments, 4^{th} Ed., Chapter 9, pp 270-341) wherein the results described in Example 1 were used to prepare combines of a select high concentration or a selected low concentration of each nutritive element as shown in Table 1.

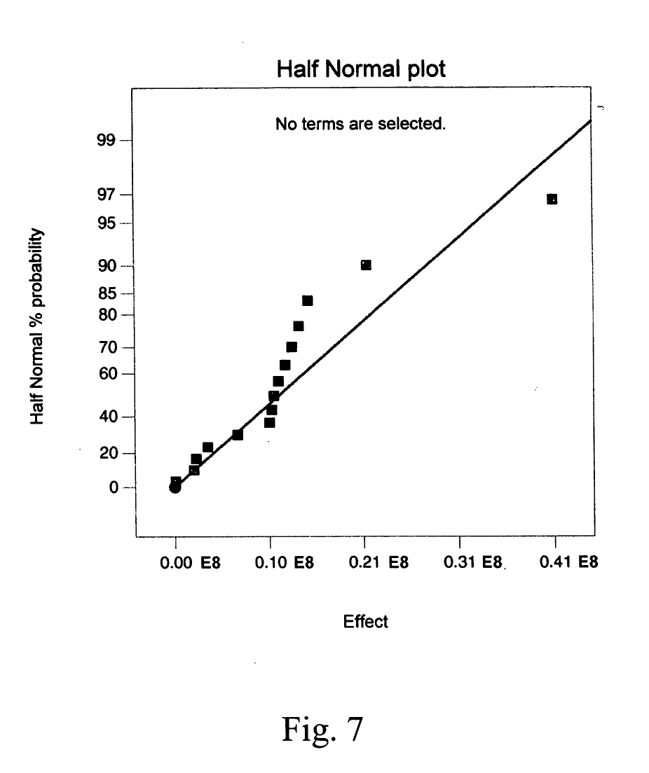

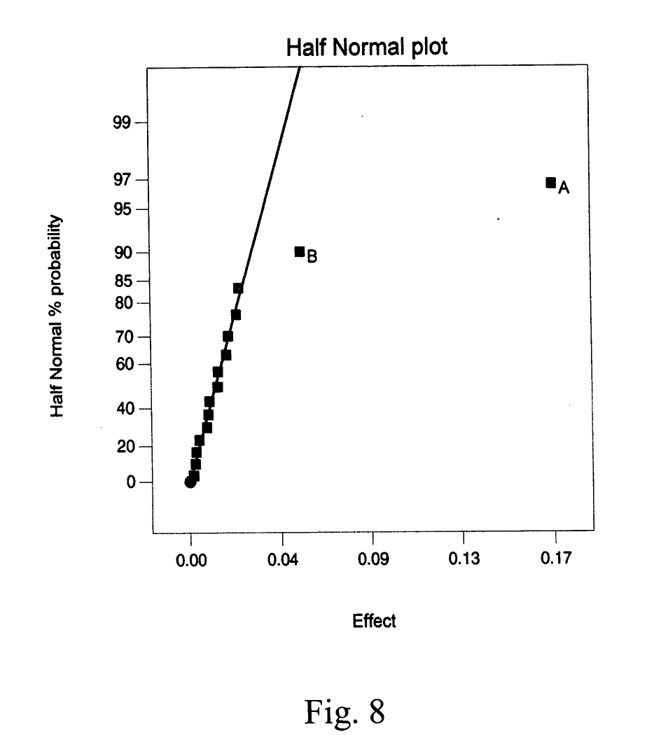

Separate batches of corn mash were prepared and supplemented with 16 mM urea after which, the individual nutrient combinations shown in Table 1 were then added to individual corn mash batches. The active dry yeast “z” was used as the inoculum in this study. Fermentation rates and yeast cell growth determinations were monitored as described in Example 1. The results were analyzed using Design-Experts version 5 (State-Ease Inc., Minneapolis, Minn.) and half-normal probability plots were generated to determine if any of the combinations of nutrients had significant effects on fermentation rate and cell yield. Based on this statistical analysis, none of the nutrient combinations had significant effects on the numbers of yeast cells produced in the urea-supplemented corn mash (FIG. 7). However, two combinations of nutrient elements significantly increased fermentation rates. First, high concentrations of (NH₄)₂HPO₄, combined with low levels of (NH₄)₂SO₄, MgSO₄.7H₂O and ZnSO₄.7H₂O significantly increased fermentation rates (FIG. 8, data point A). Second, high concentrations of (NH₄)₂SO₄ combined with low levels of (NH₄)₂HPO₄, MgSO₄.7H₂O and ZnSO₄.7H₂O (B) significantly increased fermentation rates (FIG. 8, data point B).

TABLE 1
Factor levels used for the determination of optimal levels of each
nutrient (experimental design as described in Montgomery, 1997).

Factors¹

| Experiment | P² | S³ | Mg⁴ | Zn⁵ |
|---|---|---|---|---|
| 1 | + | + | + | + |
| 2 | + | + | + | − |
| 3 | + | + | − | − |
| 4 | + | − | − | − |
| 5 | + | − | + | − |
| 6 | + | − | − | + |
| 7 | + | − | + | + |
| 8 | − | + | − | + |
| 9 | − | + | − | − |
| 10 | − | + | + | − |
| 11 | − | + | + | + |
| 12 | − | + | − | + |
| 13 | − | − | + | − |
| 14 | − | − | + | + |
| 15 | − | − | − | + |
| 16 | − | − | − | − |

¹(+) designates high concentrations and (−) designates low concentrations of each nutritional element.
²(NH₄)₂HPO₄ high concentration was 0.8 g/L and the low concentration was 0.2 g/L.
³(NH₄)₂SO₄ high concentration was 0.16 g/L and the low concentration was 0.02 g/L.
⁴MgSO₄•7H₂O high concentration was 0.197 g/L and the low concentration was 0.049 g/L.
⁵ZnSO₄•7H₂O high concentration was 2.18 mg/L and the low concentration was 0.273 mg/L.

### EXAMPLE 3

Optimization of N, P, S, Mg²⁺ and Zn²⁺ Combinations to Maximize Fermentation Rates in Corn Mash by a Commercial Active Dry Yeast.

The data in Example 2 show that combining high concentrations of phosphorus and sulfur with low concentrations of magnesium and zinc significantly stimulated fermentation rates. The Evolution Operation Method (EVOP) described by Box et al, (1969, Evolutionary Operation: A Statistical Method for Process Improvement, John Wiley & Sons, NY, N.Y.) was used to optimize the concentrations of phosphorus and sulfur combined with urea, magnesium and zinc for increasing fermentation rates in corn mash.

Multiple sterile 2-L Celstir® fermentors were each filled with 1 L of corn mash supplemented with 16 mM urea, 0.049 g/L MgSO₄.7H₂O, and 0.273 mg/L ZnSO₄.7H₂O. The active dry yeast “z” was used as the inoculum in this study. The EVOP testing was conducted by using the “low” concentration values of phosphorus and sulfur (0.8 g/L (NH₄)₂HPO₄ and 0.16 g/L (NH₄)₂SO₄) from Table 1 as the center points for assessing 4 additional phosphorus and sulphur concentrations around these points for their effects on fermentation rates. If the response of a particular point around the center point was significant, then this particular point became the new center point and 4 new concentration values would be examined as described by Box et al (1969). The EVOP protocol was followed until further increases in the concentrations tested did not have any further effects on fermentation rates. Fermentation rates were determined by repeated sampling and sample processing at specific time intervals as described in Example 1.

The data in Tables 2 and 3 show that optimal concentrations for supplementing corn mash with (NH₄)₂HPO₄ and (NH₄)₂SO₄ were 1.6 g/L and 0.32 g/L respectively when combined with 16 mM urea and 0.049 g/L MgSO₄7H₂O, and 0.273 mg/L ZnSO₄.7H₂O.

[image]

[image]

### EXAMPLE 4

Assessments of a Defined Urea Plus Salts-Based Yeast Food for Corn Mash Fermentations.

The effects of the optimized defined urea plus salts-based nutrient supplement, i.e., defined yeast food, developed in Example 3 for corn mash fermentations, were assessed by comparisons of fermentation rates and ethanol production in normal gravity corn mashes and very high gravity corn mashes that were (a) supplemented with 16 mM urea only, or (b) yeast extract, or (c) supplemented with 16 mM urea plus the defined yeast food from Example 3 which in this set of studies, contained 1.6 g/L (NH₄)₂HPO₄, 0.32 g/L (NH₄)₂SO₄, 0.049 g/L MgSO₄.7H₂O, and 0.273 mg/L ZnSO₄.7H₂O.

Normal density corn mashes were prepared as described in Example 1 and were determined to contain about 18-20 g dissolved solids per 100 mL mash as determined by density meter analysis. Very high gravity corn mash was prepared by heating 1750 mL of dH₂O containing 0.125 g of CaCl₂.2H₂O and then adding 950 g of ground corn. Then, 2.5 mL of a high temperature α-amylase (Termamyl® SC) were added to reduce viscosity in the mixture. Following the addition of the ground corn and a five-minute mixing period to produce a homogenous mixture, the temperature was increased to 96° C. where it was then held for 60 min with continual stirring of the mixture during which time, starch present in the ground corn was gelatinized. The temperature of the gelatinized starch was then reduced to 80° C. and liquefied by the incubation of a second dose of Termamyl® SC (2.5 mL) over an additional 30 min. Sterile 500 mL Celstir® fermentors were filled with 450 mL aliquots of mash. The mash thus prepared contained 30-32 g of dissolved solids/100 mL as determined by density meter analysis. The very high gravity mashes were then supplemented with 16 mM urea where needed after which, they were sterilized by the addition of 100 μL of diethyl pyrocarbonate (DEPC, Sigma Chemical Co.) per litre of mash while the mash was mechanically mixed. The sterilized mashes were then stored at 4° C. until required.

Each study was commenced by removing fermentors containing either normal gravity corn mash or very high gravity corn mash from 4° C. storage, connecting them to 30° C. water bath circulators and continually mixing the corn mash contents therein until the temperature of each mash was 30° C. Glucoamylase (1.5 mL/L) was then added to each fermentor 30 min prior to inoculation in order to initiate saccharification of the dextrins formed during gelatinization and liquefaction, after which the temperature was reduced to and maintained at 27° C. Each study consisted of three replicated treatments. The control treatment for the normal gravity corn mash was supplemented with 8 mM while the control treatment for the very high gravity corn mash was supplemented with 16 mM urea. The second treatment was the control corn mash to which was added 1% yeast extract. Yeast extract is a well-known complex nutrient supplement that is commonly used in laboratory experiments to increase fermentation rates and shorten overall fermentation times, and therefore, was used as a benchmark standard for assessments of the defined yeast food of the present invention. The third treatment was the control mash supplemented with 16 mM urea to which was added the defined urea plus salts-based nutrient supplement, i.e., yeast food, which in these studies contained 1.6 g/L (NH₄)₂HPO₄, 0.32 g/L (NH₄)₂SO₄, 0.049 g/L MgSO₄.7H₂O, and 0.273 mg/L ZnSO₄.7H₂O. The active dry yeast “z” was used as the inoculum in this study. Fermentation performance in all treatments was assessed by repeated sampling and analyses of the mashes during the fermentation periods as described in Example 1.

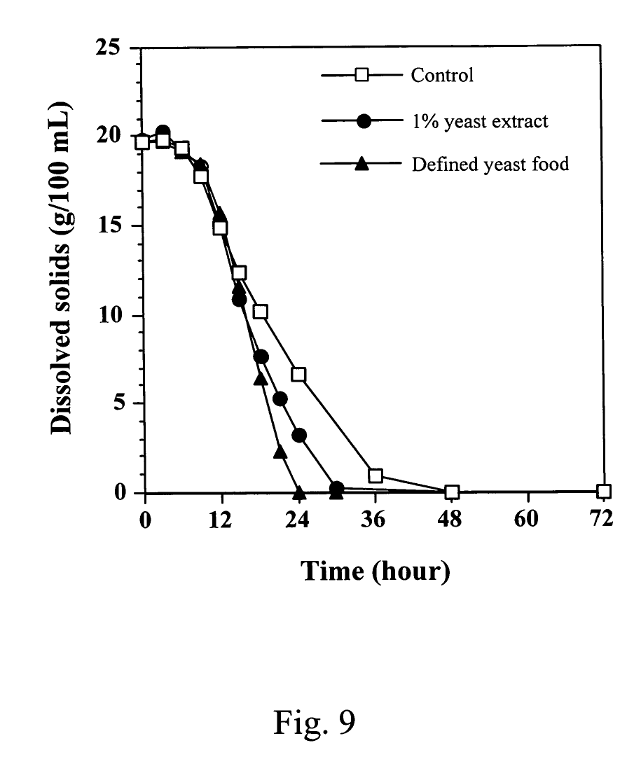

FIG. 9 shows the fermentation profiles for normal gravity corn mash supplemented with: (a) 8 mM urea, or (b) 1% yeast extract, or (c) 8 mM urea supplemented with the defined yeast food of the present invention. A fermentation period of 48 hrs was required to complete the fermentation process in the control and the yeast extract treatments, as measured by the complete disappearance of dissolved solids from the mash. However, the 1% yeast extract supplement increased the fermentation rate to 1.12 g dissolved solids/100 mL of mash from 0.79 g dissolved solids/100 mL of the mash supplemented only with 8 mM urea. However, the maximum fermentation rate in the corn mash provided with the defined yeast food of the present invention was increased to 1.59 g dissolved solids/100 mL of mash (FIG. 9). Furthermore, fermentation in corn mash containing the defined yeast food was completed in about 24 hr, i.e. in half the time that was required for mash supplemented with 8 mM urea (FIG. 9).

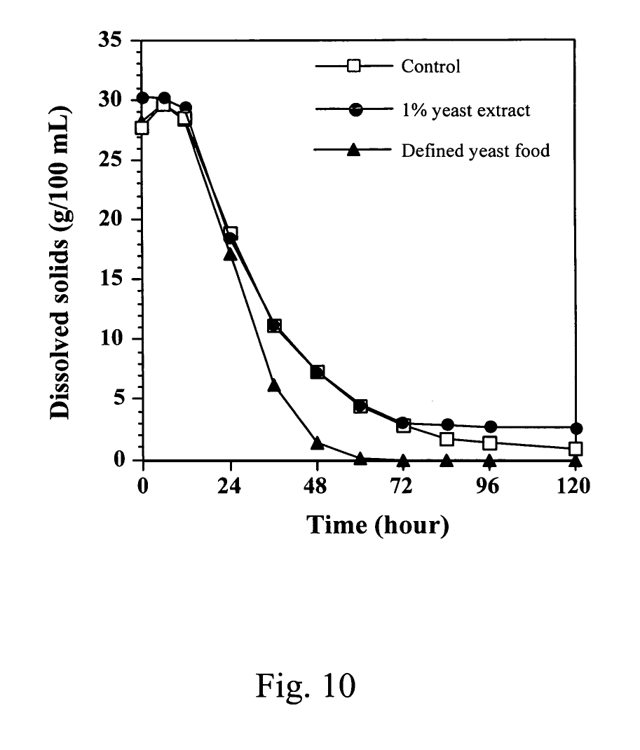

FIG. 10 shows the fermentation profiles for very high gravity corn mash supplemented with: (a) 16 mM urea, or (b) 1% yeast extract, or (c) 16 mM urea supplemented with the same defined yeast food of the present invention. After running the fermentors for 120 hr, fermentation was still not completed in either the control or the yeast extract treatments, as measured by the complete disappearance of dissolved solids from the mash. In fact, both fermentations became “stuck” after 72 hrs. However, fermentation was completed within 72 hrs after inoculation of very high gravity corn mash supplemented with 16 mM urea plus the defined yeast food of the present invention (FIG. 10). The fermentation rate in the very high gravity corn mash containing the defined yeast food was 0.928 g dissolved solids/100 mL of mash compared with the rate of 0.754 g dissolved solids/100 mL of mash supplemented with 1% yeast extract (FIG. 10).

### EXAMPLE 5

Performance of a Defined Urea Plus Salts-Based Yeast Food in a Scaled Batch Fermentation of Corn Mash.

Normal gravity corn mash was prepared as described in Example 1 with the exceptions that: (1) for preparation of the control treatment, 8 mM urea was added to the mash during the liquefaction step, and (2) for preparation of the yeast food supplemented treatment, 8 mM urea plus the defined urea plus salts-based yeast food were added during the liquefaction step. The defined urea plus salts-based yeast food used in this study contained 1.6 g/L (NH₄)₂HPO₄, 0.32 g/L (NH₄)₂SO₄, 0.049 g/L MgSO₄.7H₂O, and 0.273 mg/L ZnSO₄.7H₂O. Multiple New Brunswick Bioflow III fermentors (Edison, N.J.) were then each filled with 4 L of the designated corn mash (control or nutritionally supplemented). The active dry yeast “z” was used as the inoculum in this study. Fermentation rates and ethanol production were determined by repeated sampling and sample processing at specific time intervals as described in Example 1.

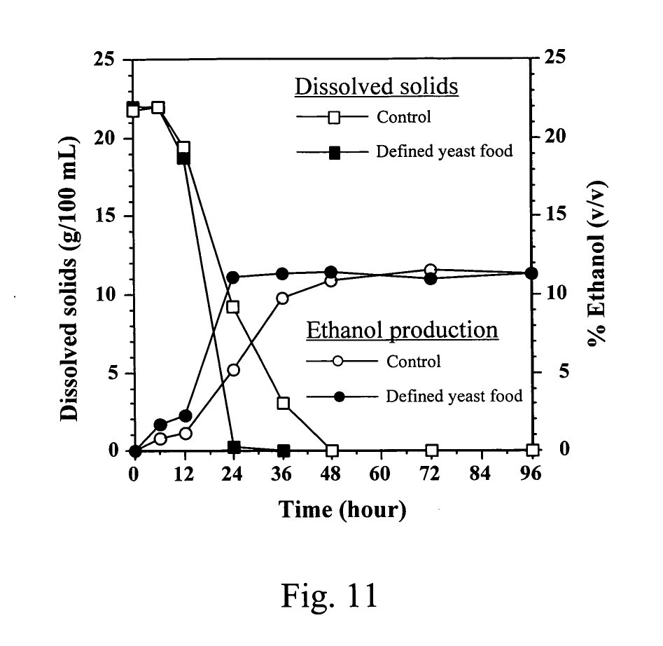

The data in FIG. 11 show that fermentation was completed within 48 hrs in the control treatment with a maximum ethanol production of 11% v/v. When the defined urea plus salts-based yeast food was added to the normal gravity corn mash, fermentations were completed within 24-36 hrs with maximum ethanol production of 11% v/v was reached within 24 hrs.

### EXAMPLE 6

Effects of a Defined Urea Plus Salts-Based Yeast Food on Fermentation Performance of Commercially Available Active Dry Yeasts.

The effects of the defined urea plus salts-based yeast food on fermentation performance of seven commercial active dry yeasts currently used by the fuel alcohol industry (Bellisimi et al., 2005, Am. Soc. Brew. Chem. J. Vol. 63, pp. 107-112) and designated herein as strains “b”, “d”, “e”, “w”, “x”, “y”, and “z”, were assessed in normal gravity corn mash prepared as described in Example 1. The commercial active dry yeasts were assessed with the following corn mash supplanted as follows: (a) 8 mM urea (control), or (b) 1% yeast extract, or (c) 8 mM urea supplemented with the defined urea plus salts yeast food of the present invention. The fermentation methods were followed as outlined in Example 4. Fermentation rates were determined by repeated sampling and sample processing at specific time intervals as described in Example 1.

The data in Table 4 show that supplementation of normal gravity corn mash with 1% yeast extract greatly increased the rates of fermentation by all seven commercial active dry yeasts tested in this study. However, supplementation of normal gravity corn with the defined urea plus salts-based yeast food of the present invention provided the greatest increases in fermentation rates for each of the strains tested. The data in Table 5 show that the defined urea plus salts-based yeast food reduced the time required for complete fermentation of corn mash by: (a) 50% for strains “w”, “x”, “z”, “b”, and “d”, and (b) more than 65% for strain “e”.

TABLE 4
Effects of nutrient supplements added to normal
gravity corn mash on fermentation performance
of commercially available active dry yeasts.

| | Fermentation rate (g/100 mL/hr) | | |
|---|---|---|---|
| Active Dry Yeast | Control | 1% yeast extract | Defined yeast food |
| “w” | 0.82 ± 0.02 | 1.02 ± 0.01 | 1.37 < 0.01 |
| “x” | 0.80 ± 0.06 | 1.28 ± 0.03 | 1.36 ± 0.02 |
| “y” | 0.83 ± 0.01 | 0.79 ± 0.08 | 0.92 ± 0.01 |
| “z” | 0.79 ± 0.01 | 1.12 ± 0.03 | 1.56 ± 0.01 |
| “b” | 0.73 ± 0.04 | 1.31 ± 0.01 | 1.37 ± 0.02 |
| “d” | 0.74 ± 0.03 | 1.37 ± 0.04 | 1.47 ± 0.01 |
| “e” | 0.67 ± 0.01 | 1.33 ± 0.05 | 1.41 ± 0.02 |

TABLE 5
Effects of nutrient supplements added to normal gravity
corn mash on the time required by commercially available
active dry yeasts to complete fermentation.

| | Fermentation time (hr)* | | |
|---|---|---|---|
| Active Dry Yeast | Control | 1% yeast extract | Defined yeast food |
| “w” | 48 | 48 | 24 |
| “x” | 48 | 30 | 24 |
| “y” | 48 | 48 | 48 |
| “z” | 48 | 48 | 24 |
| “b” | 72 | 48 | 30 |
| “d” | 48 | 30 | 24 |
| “e” | 72 | 30 | 24 |

*Time required for complete disappearance of 20 g of dissolved solids/100 mL of normal gravity corn mash.

While this invention has been described with respect to the preferred embodiments, it is to be understood that various alterations and modifications can be made to the methods for producing defined yeast food compositions comprising a mixture of selected salts, to the defined salt-based yeast food compositions of the invention described herein, and to methods for the use of the defined salt-based yeast food compositions within the scope of this invention, which are limited only by the scope of the appended claims.

1. A method for producing a defined yeast food composition for supplementing a complex fermentation feedstock, said feedstock being useful for fermentation production of ethanol therein, the method comprising:
selecting a complex fermentation feedstock;
conducting a first series of steps wherein aliquots of said feedstock are supplemented with selected concentrations of selected individual nutritive elements, said supplemented aliquots being inoculated with a yeast culture and fermentations being conducted therewith,
assessing said fermentations for effects therein of said concentrations of selected individual nutritive elements, and selecting an optimal concentration of each individual nutritive element therefrom; and
conducting a second series of steps wherein aliquots of said feedstock are supplemented with selected combinations of said individual nutritive elements, said supplemented aliquots being inoculated with a yeast culture and fermentations being conducted therewith,
assessing said fermentations for effects therein of said combinations,
selecting an optimal combination of individual nutritive elements therefrom; and
preparing a yeast food composition containing said optimal concentrations.

2. A method according to claim 1 wherein the complex fermentation feedstock is selected from the group consisting of grain mashes, pulverized vegetative plant materials, processed vegetative plant materials, ezymatically and/or chemically digested cellulosic wastestreams from wood processing operations, ezymatically and/or chemically digested lignocellulosic wastestreams from wood processing operations, high-carbohydrate-containing wastestreams from beverage production processes, high-carbohydrate-containing wastestreams from food production processes, and high-carbohydrate-containing wastestreams from industrial production processes.

3. A method according to claim 1 wherein the complex fermentation feedstock is a grain mash.

4. A method according to claim 3 wherein the grain mash is produced from a grain selected from the group consisting of corn, wheat, barley, oats, rye, triticale, and sorghum.

5. A method according to claim 4 wherein the grain mash is produced from a component processed from a grain selected from the group consisting of corn, wheat, barley, oats, rye, triticale, and sorghum.

6. A method according to claim 5 wherein said component is selected from the group consisting of purified grain, dehulled grain, fractionated grain and enriched grain.

7. A method according to claim 5 wherein said component comprises a reduced plant nutrient.

8. A method according to claim 1 wherein the complex fermentation feedstock is selected for production of fuel-grade ethanol therein.

9. A method according to claim 1 wherein the selected individual nutritive elements are selected from the group consisting of mineral salts, vitamins, enzymes, amino acids, and urea.

10. A method according to claim 9 wherein the selected individual nutritive elements are mineral salts and urea.

11. A method according to claim 10 wherein the selected individual nutritive elements are mineral elements selected from the group consisting of nitrogen, phosphorus, potassium, magnesium, sulphate, zinc, manganese, iron and trace elements.

12. A method according to claim 11 wherein the wherein the selected individual nutritive elements are mineral elements selected from the group consisting of nitrogen, phosphorus, magnesium, sulphate and zinc.

13. A method according to claim 1 wherein each selected individual nutritive element is an ionic salt containing the nutritive element, said nutritive element combined with urea.

14. A method according to claim 1 wherein the optimal combination of individual nutritive elements comprises a mixture of ionic salts and urea.

15. A method according to claim 14 wherein the optimal combination of individual nutritive elements comprises a mixture of ionic salts selected from the group consisting of nitrogen compounds, ammonium compounds, phosphate compounds, magnesium compounds, sulphate compounds, and zinc compounds, and urea.

16. A defined yeast food composition for supplementing a complex fermentation feedstock for fermentation production of ethanol therein, said yeast food composition produced according to the method of claim 1.

17. A defined yeast food composition according to claim 16, said composition comprising a mixture of urea and ionic salts.

18. A defined yeast food composition according to claim 17 wherein the mixture of ionic salts comprises salts selected from the group consisting of nitrogen salts, ammonium salts, phosphate salts, potassium salts, magnesium salts, sulphate salts, zinc salts, and trace element salts.

19. A defined yeast food composition according to claim 16 comprising urea and salts selected from the group consisting of nitrogen salts, ammonium salts, phosphate salts, potassium salts, magnesium salts, sulphate salts, zinc salts, and trace element salts.

20. A method for fermentation production of ethanol, the method comprising:
selecting a complex fermentation feedstock;
supplementing the feedstock with a defined yeast food composition produced according to claim 1;
culturing a commercial active dry yeast therein the feedstock supplemented with the yeast food composition; and
separating therefrom ethanol produced therein.

21. A method according to claim 16 wherein the complex fermentation feedstock is selected from the group consisting of grain mashes, pulverized vegetative plant materials, processed vegetative plant materials, ezymatically and/or chemically digested cellulosic wastestreams from wood processing operations, ezymatically and/or chemically digested lignocellulosic wastestreams from wood processing operations, high-carbohydrate-containing wastestreams from beverage production processes, high-carbohydrate-containing wastestreams from food production processes, and high-carbohydrate-containing wastestreams from industrial production processes.

22. A method according to claim 20 wherein the complex fermentation feedstock is a grain mash.

23. A method according to claim 22 wherein the grain mash is produced from a grain selected from the group consisting of corn, wheat, barley, oats, rye, triticale and sorghum.

24. A method according to claim 20 wherein the complex fermentation feedstock is selected for production of fuel-grade ethanol therein.

25. A method for fermentation production of ethanol, the method comprising:
selecting a complex fermentation feedstock;
supplementing the feedstock with a defined yeast food composition according to claim 15;
culturing a commercial active dry yeast therein the feedstock supplemented with the yeast food composition; and
separating therefrom ethanol produced therein.

26. A method according to claim 25 wherein the complex fermentation feedstock is selected from the group consisting of grain mashes, pulverized vegetative plant materials, processed vegetative plant materials, ezymatically and/or chemically digested cellulosic wastestreams from wood processing operations, ezymatically and/or chemically digested lignocellulosic wastestreams from wood processing operations, high-carbohydrate-containing wastestreams from beverage production processes, high-carbohydrate-containing wastestreams from food production processes, and high-carbohydrate-containing wastestreams from industrial production processes.

27. A method according to claim 25 wherein the complex fermentation feedstock is a grain mash.

28. A method according to claim 27 wherein the grain mash is produced from a grain selected from the group consisting of corn, wheat, barley, oats, rye, triticale and sorghum.

29. A method according to claim 28 wherein the grain mash is produced from a component processed from a grain selected from the group consisting of corn, wheat, barley, oats, rye, triticale, and sorghum.

30. A method according to claim 29 wherein said component is selected from the group consisting of purified grain, dehulled grain, fractionated grain and enriched grain.

31. A method according to claim 29 wherein said component comprises a reduced plant nutrient.

32. A method according to claim 24 wherein the complex fermentation feedstock is selected for production of fuel-grade ethanol therein.
